(19) United States
(12) Patent Application Publication
(10) Pub. No.: US 2021/0128515 A1
(43) Pub. Date: May 6, 2021

(54) FORMULATION OR REMEDY AGAINST ANDROGENETIC ALOPECIA

(71) Applicant: VALETUDO S.R.L. CON SOCIO UNICO, Presezzo (BG) (IT)

(72) Inventor: Vittorino BORTOLIN, Presezzo (BG) (IT)

(21) Appl. No.: 17/084,900

(22) Filed: Oct. 30, 2020

(30) Foreign Application Priority Data
Oct. 31, 2019 (IT) 102019000020118

(51) Int. Cl. A61K 31/315; A61K 9/06; A61P 17/14; A61K 31/375; A61K 31/351; A61K 31/198; A61K 47/10; A61K 47/44; A61K 47/26; A61K 47/46; A61K 47/36

(52) U.S. Cl. CPC A61K 31/315; A61K 9/06; A61P 17/14; A61K 31/375; A61K 31/351; A61K 47/36; A61K 47/10; A61K 47/44; A61K 47/26; A61K 47/46; A61K 31/198

(57) ABSTRACT

The present invention relates to a remedy against androgenetic alopecia, consisting of a combination or formulation comprising zinc and arginine gluconates combined with ascorbic acid.

### CROSS REFERENCE TO RELATED APPLICATIONS

This application claims priority to IT patent application No. 102019000020118, filed Oct. 31, 2019, which is incorporated herein by reference thereto.

### BACKGROUND OF THE INVENTION

The present invention relates, in general, to a formulation or remedy for androgenetic alopecia.

In particular, the present invention relates to such a remedy, consisting of a combination of substances or formulation.

As well as performing a protective function, hair also has an important aesthetic value, greatly contributing to the external image and self-esteem of an individual.

It is well known that many people, especially women, place considerable importance on their hair, taking care to choose the right hairstyle, care for and treat their hair, often, with considerable effort.

Hair loss is, for both sexes, an event with strong psychological repercussions, sometimes a real trauma: it is well known that the loss of hair that occurs, for example, during chemotherapy treatment is a problem that requires the use of wigs to limit the negative perception of one's condition.

Among the many causes of hair loss, the most common is by far the one involving male steroid hormones: this form of hair loss is known by the scientific name of androgenetic alopecia.

In androgenetic alopecia there is a thinning of the hair, which may be more or less marked and at a varying rate, which in men is located in the frontal-temporal area and/or tonsure area, while in women it is almost always distributed over the entire upper part of the head. The phenomenon manifests itself in men much more than in women, who generally suffer from it at an older age, usually after menopause.

Androgenetic alopecia begins with a progressive miniaturization of the hair follicle and ends with its complete atrophy. This involution of the hair follicle is caused by the presence of a substance, DHT (di-hydro-testosterone), a hormonal derivative of testosterone, which has the ability to bind to specific receptors of the hair follicle, causing its progressive deterioration. However, there is a different susceptibility of hair follicles to the action of dihydrotestosterone, greater in some areas of the scalp than in others, greater in some individuals than in others. The increased individual susceptibility of hair follicles to dihydrotestosterone is genetic: a family predisposition to androgenetic alopecia has been established.

Dihydrotestosterone is universally recognized as the hormone responsible for the progressive miniaturization of the hair follicle, and therefore for androgenetic alopecia, while testosterone as such is completely inactive in this respect.

The transformation of testosterone into di-hydro-testosterone occurs locally, in the hair follicle itself and in the attached sebaceous gland, through the intervention of a specific enzyme called 5-alpha reductase type II.

Several formulations have been tested in the past to locally inhibit the enzyme 5-alpha reductase type II, achieving some success in preventing baldness. So far, the active ingredient that has proved most effective in this respect is Finasteride, a drug used mainly systemically for the treatment of prostate cancer.

This active ingredient, although generally well tolerated by the body, nevertheless has some non-negligible side effects, such as erectile dysfunction, gynecomastia, memory loss, chronic asthenia, loss of sexual desire, muscle hypotrophy, dry skin, slowing of cognitive processes, depression, anxiety, insomnia, immune depression and others. While these drawbacks may be accepted with a certain serenity in the life-saving treatment of a tumour, it is clear that the same cannot be said for the mere improvement of a purely aesthetic condition.

It would therefore be desirable to have active ingredients able to combat androgenetic alopecia by acting locally, without inducing major side effects.

### SUMMARY OF THE INVENTION

The aim of the invention is to propose a composition of substances that overcomes the aforementioned drawbacks and that makes it possible to eliminate, or at least substantially reduce, hair loss due to androgenetic causes, without presenting the same drawbacks as Finasteride and proving, if possible, just as effective. Such purpose is achieved by a formulation against androgenetic alopecia characterised in that it comprises zinc and arginine gluconates combined with ascorbic acid, the last acting as initiator.

It should be noted that zinc and arginine gluconates have already been attributed a certain inhibitory activity against the enzyme 5-alpha reductase in the past, however not comparable with that performed by Finasteride.

The present invention, consisting of the combination of zinc and arginine gluconates with ascorbic acid, allows important advantages. In fact, this combination makes it possible to simultaneously achieve two objectives of particular importance in the fight against androgenetic alopecia, precisely:
1. the inhibition of the 5-alpha reductase type II enzyme at the hair follicle level, with effectiveness comparable, or even higher, than Finasteride;
2. increased gene expression of the SOX9 protein by hair follicle cells. This particular effect, completely unexpected and independent of the former, can lead (according to available scientific data) to a stimulation of the dermal papilla of the hair follicle and to a further contrasting action of hair miniaturization, typical of androgenetic alopecia.

### BRIEF DESCRIPTION OF THE DRAWING

The attached FIG.s illustrate the results of the in-vitro assays performed which confirm the above;

FIG. 1 shows the results of an enzyme assay on 5-alpha reductase, using Finasteride 100 nM as positive control, the values shown in the graph being the average of three different experiments, each performed in triplicate and the bars marked with asterisks representing statistically significant values ⁽*^{p}≤0.05; **^{p}<0.01 and ***^{p}≤0.0001);

FIG. 2 shows the results of an expression test of the SOX9 gene in the dermal papilla. The values reported are the average of three different experiments, each of them performed in triplicate and the bars marked with asterisks representing statistically significant values ⁽*^{p}≤0.05; **^{P}≤0.01 and ***^{p}≤0,0001). In this case, the Transformer Growth Factor beta type (TGF β) was used as the reference standard.

Below is a description of how the two tests mentioned above were performed:

Table 1 below shows the substances tested.

ZnGl stands for zinc gluconate, AA stands for ascorbic acid and ARG stands for arginine gluconate.

TABLE 1

| Sample no. | Compounds | Physical state |
|---|---|---|
| 1 | ARG | liquid |
| 2 | ZnGl | liquid |
| 3 | ZnGlARG | liquid |
| 4 | ARGAA | liquid + powder |
| 5 | ZnGlAA | liquid + powder |
| 6 | ZnGlARGAA | liquid + powder |

### DETAILED DESCRIPTION OF PREFERRED EMBODIMENTS

### 5-Alpha Reductase Type II Enzyme Assay

8×10³ dermal papilla cells of the human follicle are seeded in 96 well plates and treated with the compounds to be examined and with 100 nM testosterone for 24 hours.

Coverage of 20 ng/ml of BSA-DHT at 4° C. in 100 μl of 50 mM sodium carbonate, pH 9.0. is performed.

After washing in PBS, the plate containing BSA-DHT is incubated with 50 μl of cell supernatant, to which 50 μl of biotin-conjugated primary anti-DHT antibody is added, dissolved in PBS containing 1% by weight of BSA.

The plate is washed three times in PBS two hours later and incubated with 100 μl of streptavidin-HRP 5 μg/ml in PBS containing 1% by weight of BSA.

The amount of DHT is measured by colorimetric reaction, using a solution of 0.5 mg/ml OPD at 0.012% by weight.

The plate is incubated at room temperature until colour development and the absorbance of the sample is measured at 490 nm with a Perkin Elmer Victor3 plate reader.

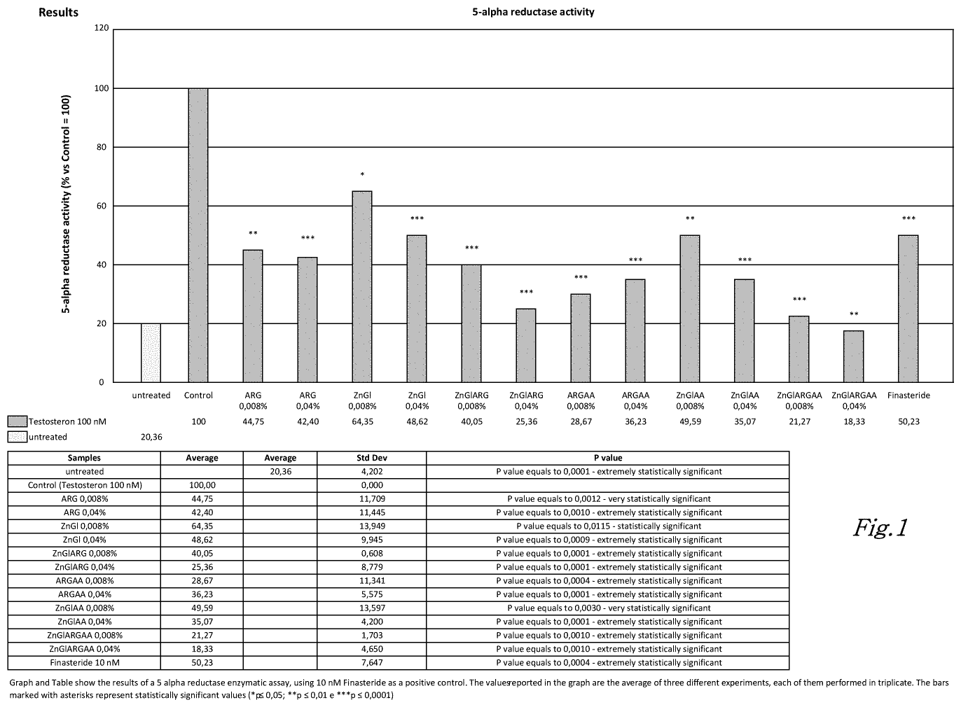

The results are shown in FIG. 1.

These results show that the ZnGl-ARG-AA combination, which is the subject of this invention, gives the best results, superior to those of Finasteride itself, used as a reference standard.

### Assay on the Expression of the Sox9 Gene in the Dermal Papilla

To analyse gene expression in basal conditions, 10⁶ dermal papilla cells from the human follicle are sown in 6 well plates, incubated for 24 hours with test compounds, and then processed for total RNA extraction. Total RNA is extracted with a GenElute Mammalian Total RNA Purification Kit (Sigma), according to the manufacturer's instructions. Everything is treated with DNase I at 37° C. for 30 minutes to eliminate any contaminating genomic DNA.

The first strand cDNA is synthesized from 0.5-1 μg, using the RevertAid First Strand cDNA Synthesis Kit (Fermentas). RT-PCR is performed at room temperature (TA), using gene specific primers and the QuantumRNA 18S internal standard (Ambion) according to manufacturer's instructions. The QuantumRNA kit contains primers to amplify 18S rRNA along with competimers that reduce the amplified 18S rRNA product within the range to allow it to be used as endogenous standard.

The amplification reactions are performed with the following general scheme: 2 min at 94° C. followed by 35 cycles of 94° C. for 30 s, annealing temperature (specific for each gene) for 30 s, and 72° C. for 30-60 s, with a 10 min final extension at 72° C.

The PCR products obtained are loaded on 1.5% agarose gel, and the amplification bands are visualized and quantified with the Geliance 200 Imaging system (Perkin Elmer). The amplification band corresponding to the gene analysed is normalized to the amplification band corresponding to the 18S. The values obtained are finally converted into percentage values by considering the measure of the untreated controls as 100%.

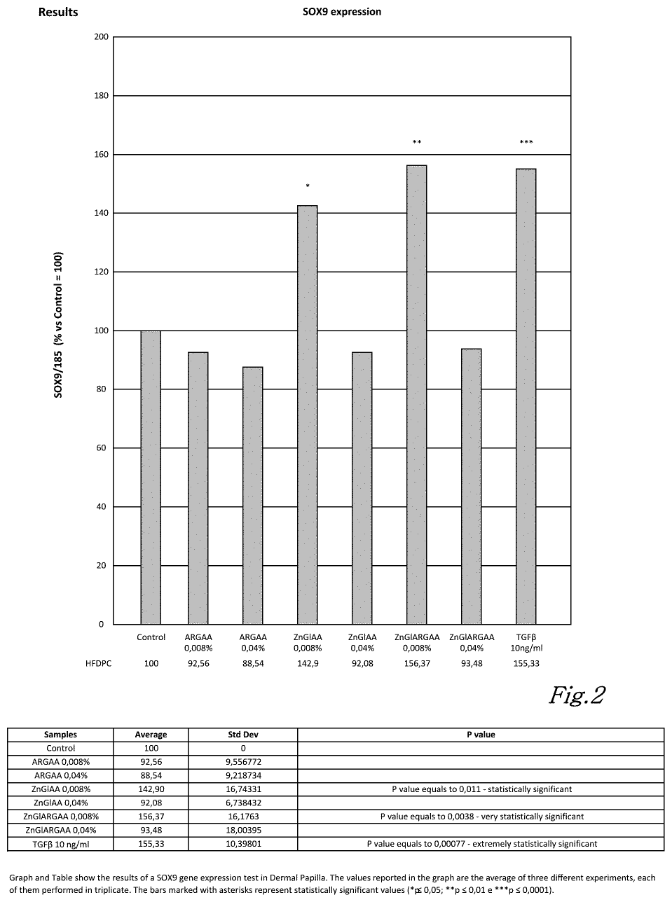

The results are shown in FIG. 2.

These results show that the ZnGl-ARG-AA combination, which is the subject of this invention, gives the best results, comparable to those obtained with TGF β□ used as a reference standard.

The present invention, as we have just seen, consists of a formulation containing zinc and arginine gluconates combined with ascorbic acid, to be used against androgenetic alopecia, acting primarily by inhibiting the enzyme 5-alpha reductase type II, and secondarily by inducing the SOX9 protein.

The formulation according to the present invention consists of two separate phases, to be mixed at the moment of use, this being a fundamental requirement to guarantee the stability over time of the components and therefore their respective efficacy: the first phase can be liquid or creamy or in the form of gel and contains zinc and arginine gluconates and relative adjuvants and excipients, while the second phase can be in the form of powder or suspension of solid particles in an oily or otherwise anhydrous vehicle and contains ascorbic acid and relative adjuvants and excipients.

The final preparation resulting from the union of the two aforementioned phases can be used for topical applications on the scalp according to different methods, such as massages, brushing, subcutaneous injections, sprays, shampoos or even through technologies and equipment of all kinds, the latter typically chosen in the group including equipment for skin stimulation with electromagnetic pulses, equipment for phototherapy using laser light.

The term “relative adjuvants and excipients” of zinc gluconates and arginine or ascorbic acid according to the present invention means at least one compound chosen from the group comprising Niacinamide Ascorbate, Ascorbyl Retinoate, Vanillyl Butyl Ether, Menthol, Anethole, Ellagic Acid, Panthenol, Tocopherol, Tocopheryl Acetate, Citric Acid, Lipoic Acid, Lecithin, Magnesium Ascorbate, Magnesium Ascorbyl Phosphate, Sodium Ascorbate, Sodium Ascorbyl Phosphate, Calcium Ascorbate, Ascorbyl Tetraisopalmitate, 3-O Ethyl Ascorbic Acid, Ascorbyl Glucoside, Glyceryl Octyl Ascorbic Acid, Tetrahexyldecyl Ascorbate.

According to a particularly advantageous embodiment of the present invention, it is expected that arginine gluconate, an essential component of the present invention, can be obtained during production starting from arginine base, salifying the same with stoichiometric quantities of gluconic acid or the relative precursor gluconolactone.

The formulation may comprise the further addition of arginine in the form of a free, non-salified base.

It is also possible that the formulation according to the present invention contains additional amounts of gluconic acid or gluconolactone.

Advantageously, the formulation envisages that the final preparation intended for application is obtained at the time of use or at a time very close to such use, since the formulation is not very stable and tends to degrade after about four days. Advantageously, the formulation according to the present invention is sold as a kit, consisting of two separate phases, packaged in two different compartments, to be mixed just before use, each of which contains some components of the overall formula to the exclusion of others and vice versa.

The formulation according to the present invention may include other ingredients such as excipients, preservatives, stabilizers, solvents, charges, fillers, and the like typically chosen from the group comprising Ethyl Alcohol, Isopropyl Alcohol, Propylene Glycol, Butylene Glycol, Glycerin, Polysorbate 20, PEG-40 Hydrogenated Castor Oil, Laureth-9, Dimethicone, Silica, Magnesium Stearate, Piroctone Olamine, Cyclopirox Olamine.

It may also contain synergistic adjuvants or compounds, known in themselves, typically chosen from the group comprising Soy Isoflavones, Genistein, Serenoa Serrulata Fruit Extract, Minoxidil.

Without being bound by theory, the formulation of the present invention has an effect on 5-alpha reductase type II, acting like Finasteride, but without presenting the same undesirable side effects. It also has the ability to increase the gene expression of the transcription factor SOX9, a protein capable of stimulating the dermal papilla of the hair follicle, counteracting the progressive miniaturisation of the same through a mechanism of action different from the previous and synergistic with it.

In order to better describe the present invention, some examples are given, which describe only some of the possible compositions of the invention. Although such examples use selected formulations in accordance with this invention, it is clear that such examples are illustrative only and not limiting.

All the components of the compositions of this invention are commercially available or can be easily prepared following procedures known in the art.

All parts, percentages and proportions referred to and indicated in the claims are understood to be the total weight of the composition, unless otherwise stated.

The names of the ingredients used and shown in the examples follow the International Nomenclature of Cosmetic Ingredients (INCI) and are those officially recognized in the cosmetic sector.

The ingredient “Aqua” mentioned in the examples below is always deionised water.

### Example 1: Two-Phase Hair Loss Prevention Lotion for Men (the Two Phases are Divided into Separate Compartments and Combined at the Time of Use by Mixing them Thoroughly)—by Weight

Phase A (to be placed in the main compartment): Aqua 59.70% Zinc Gluconate 0.90% Arginine 0.65% Gluconic Acid 1.20% Alcohol denat. 22.40% Serenoa Serrulata Fruit Extract 2.00% Propylene Glycol 1.50% Polysorbate-20 0.75% Menthol 0.15% Vanillyl Butyl Ether 0.05%
Part B (to be placed in the secondary compartment) Ascorbic Acid 10.00% Magnesium Stearate 0.70%

Preparation:
I) prepare part A (liquid) by simple mixing of the ingredients under constant stirring
(II) prepare Part B (solid) by careful and thorough mixing of the two fine powder ingredients composing it
(III) separately place part A in the main compartment (vial) and part B in the secondary compartment (cap-reservoir).

### Example 2: Two-Phase Hair Loss Prevention Lotion for Women (the Two Phases are Divided into Separate Compartments and Combined at the Time of Use by Mixing them Thoroughly)—by Weight

Phase A (to be placed in the main compartment): Aqua 64.95% Zinc Gluconate 0.80% Arginine 0.55% Gluconic Acid 0.50% Gluconolactone 0.45% Alcohol denat. 21.20% Propylene Glycol 1.50% Polysorbate-20 0.75% Menthol 0.15% Polysorbate-80 0.15% Lecithin 0.15% Soy Isoflavones 0.10% Vanillyl Butyl Ether 0.05%
Part B (to be placed in the secondary compartment) Ascorbic Acid 8.00% Magnesium Stearate 0.70%

Preparation:
I) prepare part A (liquid) by simple mixing of the ingredients under constant stirring
(II) prepare Part B (solid) by careful and thorough mixing of the two fine powder ingredients composing it
(III) separately place part A in the main compartment (vial) and part B in the secondary compartment (cap-reservoir).

### Example 3: Two-Phase Scalp Cream Mask for Men (the Two Phases are Divided into Separate Compartments and Combined at the Time of Use by Mixing them Thoroughly)—% by Weight

Phase A (to be placed in the main compartment): Aqua 76.80% Zinc Gluconate 0.90% Arginine 0.75% Gluconic Acid 0.70% Gluconolactone 0.35% Serenoa Serrulata Fruit Extract 2.50% PEG-40 Castor Oil 1.60% Propylene Glycol 1.50% Polysorbate-20 0.90%
Part B (to be placed in the secondary compartment) Ascorbic Acid 8.75% Cocoglycerides 2.50% Isopropyl Myristate 2.50% Menthol 0.20% Vanillyl Butyl Ether 0.05%

Preparation:
I) prepare part A (liquid) by simple mixing of the ingredients under constant stirring
(II) prepare Part B (suspension of solids in liquid) by careful mixing of the ingredients which compose it
(III) separately place part A in the main compartment (vial) and part B in the secondary compartment (cap-reservoir, tube or vial).

### Example 4: Two-Phase Scalp Cream Mask for Women (the Two Phases are Divided into Separate Compartments and Combined at the Time of Use by Mixing them Thoroughly)—% by Weight

Phase A (to be placed in the main compartment): Aqua 81.40% Zinc Gluconate 0.80% Arginine 0.65% Gluconic Acid 0.50% Gluconolactone 0.45% PEG-40 Castor Oil 1.50% Propylene Glycol 1.50% Menthol 0.15% Polysorbate-80 0.15%
Part B (to be placed in the secondary compartment) Ascorbic Acid 7.75% Cocoglycerides 2.50% Isopropyl Myristate 2.50% Genistein 0.10% Vanillyl Butyl Ether 0.05%.

Preparation:
I) prepare part A (liquid) by simple mixing of the ingredients under constant stirring
(II) prepare Part B (suspension of solids in liquid) by careful mixing of the ingredients which compose it
(III) separately place part A in the main compartment (vial) and part B in the secondary compartment (cap-reservoir, tube or vial).

### Example 5: Unisex, Two-Phase Hair Loss Prevention Lotion (the Two Phases are Divided into Separate Compartments and Combined at the Time of Use by Mixing them Thoroughly)—% by Weight

Phase A (to be placed in the main compartment): Aqua 13.00% Zinc Gluconate 0.20% Arginine 0.15% Gluconolactone 0.15% Alcohol denat. 30.00% Propylene Glycol 50%
Part B (to be placed in the secondary compartment) Ascorbic Acid 3.00% Minoxidil 3.00% Silica 0.25% Magnesium Stearate 0.25%

Preparation:
I) prepare part A (liquid) by simple mixing of the ingredients under constant stirring
(II) prepare Part B (solid) by careful and thorough mixing of the four fine powder ingredients composing it
(III) separately place part A in the main compartment (vial) and part B in the secondary compartment (cap-reservoir).

It is understood, however, that the invention is not to be considered limited to the particular arrangement illustrated above, which is merely an embodiment provided by way of example, but that various variations are possible, all within the reach of a person skilled in the art, while remaining within the scope of protection of the invention itself, as defined by the following claims.

1) Formulation against androgenetic alopecia characterized in that said formulation comprises zinc and arginine gluconates combined with ascorbic acid, said ascorbic acid acting as initiator.

2) The formulation according to claim 1), characterized in that the formulation further comprises gluconic acid or gluconolactone.

3) The formulation against androgenetic alopecia according to claim 1), characterized in that the formulation comprising zinc and arginine gluconates combined with ascorbic acid primarily acts through the inhibition of the 5-alpha reductase type II enzyme and secondarily through the induction of the SOX9 protein.

4) The formulation according to claim 1) wherein the arginine gluconate, essential component of the formulation of claim 1, is obtained during production starting from arginine base and salifying it with stoichiometric amounts of gluconic acid or of the relative precursor, the gluconolactone.

5) The formulation according to claim 1), characterized in that said formulation comprises the further addition of arginine in the form of free base, not salified.

6) The formulation according to claim 1), characterized in that said formulation comprises other ingredients such as excipients, preservatives, stabilizers, solvents, charges, fillers and the like, as well as adjuvants or synergistic compounds belonging to the state of the art.

7) The formulation according to claim 1), characterized in that said formulation consists of two separate phases, to be mixed at the time of use or at a time very close to such use, to ensure the stability of the components over time and therefore the respective effectiveness.

8) The formulation according to claim 7), characterized in that said first phase of the formulation is liquid or creamy or in the form of a gel and contains zinc and arginine gluconates and relative adjuvants and excipients, and said second phase is in form of powder or suspension of solid particles in an oily or anyway anhydrous vehicle and contains ascorbic acid and relative adjuvants and excipients.

9) The formulation according to claim 7), characterized in that the final preparation resulting from the union of said two phases is used for topical applications on the scalp according to methods of different types such as massages, brushing, sub-cutaneous injections, sprays, shampoos or even through technologies and equipment of all kinds.

10) The formulation according to claim 7), characterized in that said formulation is marketed as a kit consisting of two separate phases or parts, packaged in two different compartments, main and secondary compartments, to be mixed just before use, each of them contains some components of the overall formula, with the exclusion of other components.

11) The formulation according to claim 8), characterized in that said first phase, to be partitioned in said main compartment, contains, in percentage by weight: water from 50.00 to 90.00%, zinc gluconate from 0.10 to 6.00%, arginine from 0.20 to 12.00%, gluconic acid or gluconolactone from 0.20 to 12.00%, and in that said second phase, to be partitioned in said secondary compartment, contains ascorbic acid from 0.50 to 15.00%; with the clarification that the aforementioned percentages are to be understood as calculated with respect to the total of the 2 phases.

12) Method of preparation of the formulation according to claim 7), characterized in that the first liquid phase is prepared by simple mixing of the ingredients under constant stirring; the second phase—which is solid or in suspension of solid in liquid—is prepared through careful and prolonged mixing of the ingredients that compose it; the method further characterized in that the first phase in the main vial compartment and the second phase in the secondary tank cap or tube or vial compartment are separately partitioned.
